(19) United States
(12) Patent Application Publication
(10) Pub. No.: US 2019/0183137 A1
(43) Pub. Date: Jun. 20, 2019

(54) METHODS FOR PASTEURIZING AND/OR STERILIZING PARTICULATE GOODS

(71) Applicant: BÜHLER AG, Uzwil (CH)

(72) Inventors: Nicolas MENESES, Gossau (CH); Martin HERSCHE, St. Gallen (CH); Alasdair CURRIE, London (GB); Niklaus SCHÖNENBERGER, Herisau (CH); Thomas SCHEIWILLER, Zuzwil (CH)

(21) Appl. No.: 16/326,808

(22) PCT Filed: Aug. 17, 2017
(86) PCT No.: PCT/EP2017/070843

§ 371 Date: Feb. 20, 2019

(30) Foreign Application Priority Data
Aug. 20, 2016 (EP) 16185056.5

(51) Int. Cl. A23B 9/06; A23L 3/26; A61L 2/08; B65B 37/04; B65B 55/16

(52) U.S. Cl. CPC A23B 9/06; A23L 3/263; B65B 55/16; B65B 37/04; A61L 2/087

(57) ABSTRACT

Methods for pasteurizing and/or sterilizing particulate goods (1), containing the following steps: a) producing an electron beam (5), b) pasteurizing and/or sterilizing the goods (1) by the electron beam (5) in a treatment zone (3), wherein the electrons of the electron beam (5) have an energy that lies in the range of 80 keV to 300 keV, preferably from 140 keV to 280 keV, more preferably from 180 keV to 260 keV, the goods (1) being exposed to the electron beam (5) for a treatment time which lies in the range from 5 ms to 25 ms, and the electron beam (5) has a mean electron current density in the treatment zone (3) which lies in the range of 10¹⁵ s⁻¹·cm² to 2.77·10¹⁵ s⁻¹·cm⁻².

The present invention concerns methods for pasteurizing and/or sterilizing particulate goods by means of an electron beam.

Particulate goods are defined here and in the following as goods consisting inter alia of grains and/or flakes, whereby the particles can have a spherical, plate-shaped or angular shape. They may also be ground particles. Pasteurization and/or sterilization, for example, can kill or render harmless at least the majority of micro-organisms. In particular, a reduction of harmful microorganisms by at least five orders of magnitude can be achieved.

A device for pasteurizing and/or sterilizing particulate goods is known for example from EP 1 080 623 B1. This device contains vibrating conveyors with which seed can be separated into a transparent curtain. This curtain is then guided through an electron field generated by an electron accelerator which can, for example, sterilize the seed.

Another device known from the U.S. Pat. No. 5,801,387 A is used for pasteurizing and/or sterilizing particulate goods. In the device according to that invention, a particle-shaped good is dosed into a horizontal air stream with a vibration conveyor and then exposed to an electron beam. A vacuum pump and a filter are then used to classify the goods.

Furthermore, the DE 10 2012 209 434 A1 reveals a device that separates and rotates a free-flowing product with the aid of a vibration conveyor and a rotating brush roller. The particles then pass in free fall through an electron field.

EP 0 513 135 B1 discloses a device with which seed is introduced into a vertical chute by means of rotary valves, where it is subjected in vertical fall to electron beams.

Another device known from EP 0 705 531 B1 is a dosing device which introduces the seed into a process chamber by means of an unspecified dosing device, in which it falls vertically through an electron beam.

The device disclosed in U.S. Pat. No. 6,486,481 B1 contains a vibrating table on which a polymeric good is moved and exposed to an electron beam. However, this is not done for pasteurisation or sterilization purposes, but to reduce the molecular weight of the polymeric good.

It is an aim of the present invention to overcome the disadvantages known from the prior art. In particular, methods are to be provided with which particulate goods can be pasteurised and/or sterilised effectively, reliably and as simply, quickly and inexpensively as possible.

These and other tasks are solved by the method according to the present invention of pasteurizing and/or sterilizing particulate goods. It comprises the following steps:
a) Generating an electron beam,
b) Pasteurisation and/or sterilisation of the good using the electron beam in a treatment zone.

According to the invention, the electrons of the electron beam have an energy in the range from 80 keV to 300 keV, preferably from 140 keV to 280 keV, more preferably from 180 keV to 260 keV. Lower electron energies would not produce sufficient pasteurization and/or sterilization. Higher electron energies could not achieve significantly higher degrees of pasteurization and/or sterilization.

Furthermore, according to the invention, the electron current density in the treatment zone is in the range of 10¹⁵ s⁻¹·cm⁻² to 2.77·10¹⁵ s⁻¹·cm⁻². In this range, sufficient pasteurization and/or sterilization is achieved.

According to the invention, the good is also exposed to the electron beam for a treatment time in the range of 5 ms to 25 ms. A certain minimum treatment time is required for sufficient pasteurisation and/or sterilisation. Too long treatment times have not shown a significantly increased degree of pasteurisation and/or sterilisation.

The product can be a foodstuff such as cereals such as soya, breakfast cereals, snacks, nuts such as dried coconuts, almonds, peanut butter, cocoa beans, chocolate, chocolate liquid, chocolate powder, chocolate chips, cocoa products, pulses, coffee, seeds such as pumpkin seeds, spices (such as turmeric, particularly in slices), tea mixtures, dried fruit, pistachios, dry protein products, bakery products, sugar, potato products, pasta, baby food, dried egg products, soya products such as soya beans, thickeners, yeasts, yeast extracts, gelatine or enzymes.

Alternatively, the product may also be a pet food, such as pellets, feed for ruminants, poultry, aquatic animals (in particular fish) or pets, or compound feed.

It is, however, also conceivable and lies within the scope of the invention that the good is, for example, a plastic such as PET, for example in the form of flakes or pellets.

The electron beam is advantageously used to expose the good to a radiation dose in the range from 1 kGy to 45 kGy, preferably from 8 kGy to 30 kGy, especially preferred from 10 kGy to 16 kGy.

It is advantageous to separate the good before the treatment in step b). This separation ensures that each individual grain of the good is captured by the electron beam and thus pasteurised and/or sterilised. Separation can be achieved, for example, with the aid of vibrating surfaces which are excited to vibrate and which optionally have one or more channels. Alternatively or additionally a separation can be achieved by a sliding surface on which the good slides down.

Furthermore, it is advantageous that the good falls freely through the treatment zone. The good is called “free-falling” if the trajectories of the individual particles of the good are determined solely by their velocity, the force of gravity acting on them and, if applicable, a process gas surrounding the good. In particular, the particles of the good do not slide on a surface through the treatment zone. In free fall, the speed is independent of the throughput, so that throughputs in the range of 100 kg/h to 1000 kg/h can be achieved at the same speed.

For many goods, in particular for a large number of spices, it has proven to be advantageous if the goods move through the treatment zone at a speed ranging from 1 m/s to 5 m/s, preferably from 2 m/s to 4 m/s, particularly preferably from 2 m/s to 3 m/s. The speed of the goods is determined by the speed of the spices. The higher the speed of the goods, the higher the achievable throughput. On the other hand, the speeds must not be too high so that the goods remains in the electron beam long enough to be pasteurized and/or sterilized.

In the following, the invention is explained in more detail by way of specific embodiments and drawings.

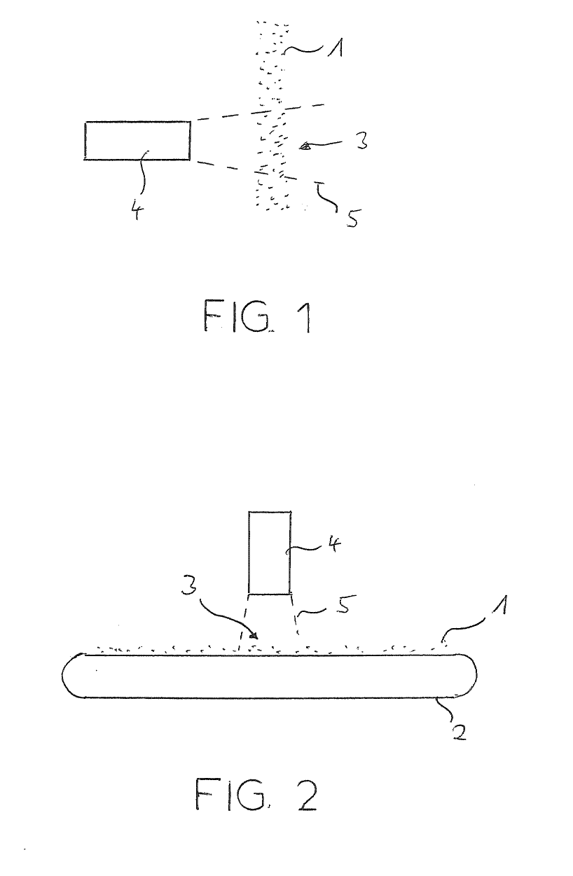

FIG. 1: a schematic representation of a first method according to the invention;

FIG. 2: a schematic representation of a second method according to the invention.

In the first embodiment schematically shown in FIG. 1, a particulate, separated good 1, such as a spice, pistachios or almonds, falls freely through a treatment zone 3 at an increasing speed in the range from 1 m/s to 5 m/s. There it is pasteurized and/or sterilized by means of an electron beam generated by an electron source 4. The electron beam contains electrons of an energy in the range 80 keV to 300 keV and has an average electron current density in the treatment zone 3 in the range of 10¹⁵ s⁻¹·cm⁻² to 2.77·10¹⁵ s⁻¹·cm⁻². The good 1 is subjected to this treatment for a treatment time in the range of 5 ms to 25 ms, whereby it is exposed to a radiation dose in the range 1 kGy to 45 kGy.

FIG. 2 schematically shows a second embodiment. A separated particulate good 1 is dosed onto a conveyor belt 2. The conveyor belt 2 transports the good 1 in a treatment zone 3 under an electron source 4. In treatment zone 3, the electron source 4 generates an electron beam with electrons of an energy in the range from 80 keV to 300 keV and an average electron current density in the range from 10¹⁵ s⁻¹·cm⁻² to 2.77·10¹⁵ s⁻¹·cm⁻². The good 1 is subjected to this treatment for a treatment time in the range from 5 ms to 25 ms, whereby it is exposed to a radiation dose in the range from 1 kGy to 45 kGy.

With these methods, the particulate good 1 can be pasteurized and/or sterilized effectively and reliably, but still as simply, quickly and inexpensively as possible.

1.-6. (canceled)

7. A method for pasteurizing and/or sterilizing particulate material (1), containing the following steps:
a) producing an electron beam (5),
b) pasteurizing and/or sterilizing the material (1) by the electron beam (5) in a treatment zone (3), wherein
the electrons of the electron beam (5) have energy that lies in a range of from 80 keV to 300 keV,
the electron beam (5) in the treatment zone (3) has a mean electron current density that lies in a range of from 10¹⁵ s⁻¹·cm⁻² to 2.77·10¹⁵ s⁻¹·cm²,
the material (1) is exposed to the electron beam (5) for a treatment time that lies in a range of from 5 ms to 25 ms.

8. The method according to claim 7, wherein the electrons of the electron beam (5) have energy that lies in the range of from 140 keV to 280 keV.

9. The method according to claim 7, wherein the electrons of the electron beam (5) have energy that lies in the range of from 180 keV to 260 keV.

10. The method according to claim 7, wherein the material (1), via the electron beam (5), is exposed to a dose of radiation that lies in a range of from 1 kGy to 45 kGy.

11. The method according to claim 7, wherein the material (1), via the electron beam (5), is exposed to a dose of radiation that lies in a range of from 8 kGy to 30 kGy.

12. The method according to claim 7, wherein the material (1), via the electron beam (5), is exposed to a dose of radiation that lies in a range of from 10 kGy to 16 kGy.

13. The method according to claim 7, wherein the material (1) is separated prior to step b).

14. The method according to claim 10, wherein the material (1) is separated prior to step b).

15. The method according to claim 7, wherein the material (1) falls freely through the treatment zone (3).

16. The method according to claim 10, wherein the material (1) falls freely through the treatment zone (3).

17. The method according to claim 13, wherein the material (1) falls freely through the treatment zone (3).

18. The method according to claim 14, wherein the material (1) falls freely through the treatment zone (3).

19. The method according to claim 7, wherein the material (1) is moved through the treatment zone (3) at a speed (v) that lies in a range of from 1 m/s to 5 m/s.

20. The method according to claim 7, wherein the material (1) is moved through the treatment zone (3) at a speed (v) that lies in a range of from 2 m/s to 4 m/s.

21. The method according to claim 7, wherein the material (1) is moved through the treatment zone (3) at a speed (v) that lies in a range of from 2 m/s to 3 m/s.

22. The method according to claim 7, wherein the material (1) is selected from the group consisting of:
foodstuffs, such as grains, for example soya, breakfast cereals, snacks, nuts, such as dried coconut, almonds, peanut butter, cocoa beans, chocolate, liquid chocolate, chocolate powder, chocolate chips, cocoa products, pulses, coffee, seeds, for example pumpkin seeds, spices (such as turmeric, in particular sliced), tea mixtures, dried fruits, pistachios, dried protein products, bakery products, sugar, potato products, pasta products, baby food, dried egg products, soya products, for example soya beans, thickeners, yeasts, yeast extracts, gelatins or enzymes;
animal feed, such as pellets, food for ruminants, birds, aquatic species (in particular fish) or domestic pets, or
compound feed;
plastics, such as PET, for example in the form of flakes or pellets.

23. The method according to claim 10, wherein the material (1) is selected from the group consisting of:
foodstuffs, such as grains, for example soya, breakfast cereals, snacks, nuts, such as dried coconut, almonds, peanut butter, cocoa beans, chocolate, liquid chocolate, chocolate powder, chocolate chips, cocoa products, pulses, coffee, seeds, for example pumpkin seeds, spices (such as turmeric, in particular sliced), tea mixtures, dried fruits, pistachios, dried protein products, bakery products, sugar, potato products, pasta products, baby food, dried egg products, soya products, for example soya beans, thickeners, yeasts, yeast extracts, gelatins or enzymes;
animal feed, such as pellets, food for ruminants, birds, aquatic species (in particular fish) or domestic pets, or
compound feed;
plastics, such as PET, for example in the form of flakes or pellets.

24. The method according to claim 13, wherein the material (1) is selected from the group consisting of:
foodstuffs, such as grains, for example soya, breakfast cereals, snacks, nuts, such as dried coconut, almonds, peanut butter, cocoa beans, chocolate, liquid chocolate, chocolate powder, chocolate chips, cocoa products, pulses, coffee, seeds, for example pumpkin seeds, spices (such as turmeric, in particular sliced), tea mixtures, dried fruits, pistachios, dried protein products, bakery products, sugar, potato products, pasta products, baby food, dried egg products, soya products, for example soya beans, thickeners, yeasts, yeast extracts, gelatins or enzymes;
animal feed, such as pellets, food for ruminants, birds, aquatic species (in particular fish) or domestic pets, or
compound feed;
plastics, such as PET, for example in the form of flakes or pellets.

25. The method according to claim 14, wherein the material (1) is selected from the group consisting of:
foodstuffs, such as grains, for example soya, breakfast cereals, snacks, nuts, such as dried coconut, almonds, peanut butter, cocoa beans, chocolate, liquid chocolate, chocolate powder, chocolate chips, cocoa products, pulses, coffee, seeds, for example pumpkin seeds, spices (such as turmeric, in particular sliced), tea mixtures, dried fruits, pistachios, dried protein products, bakery products, sugar, potato products, pasta products, baby food, dried egg products, soya products, for example soya beans, thickeners, yeasts, yeast extracts, gelatins or enzymes;
animal feed, such as pellets, food for ruminants, birds, aquatic species (in particular fish) or domestic pets, or
compound feed;
plastics, such as PET, for example in the form of flakes or pellets.
